(19) United States
(12) Patent Application Publication
(10) Pub. No.: US 2026/0021221 A1
(43) Pub. Date: Jan. 22, 2026

(54) MIXING AND DELIVERY DEVICES FOR TOPICAL SKIN ADHESIVES, CONTAINING ANTIMICROBIAL AGENTS INSIDE THE DEVICE EXPRESSION TIP OR STATIC MIXER

(71) Applicant: Ethicon, Inc., Raritan, NJ (US)

(72) Inventor: DUAN LI OU, Warren, NJ (US)

(21) Appl. No.: 18/779,273

(22) Filed: Jul. 22, 2024

(51) Int. Cl. A61L 26/00; A61B 17/00

(52) U.S. Cl. CPC A61L 26/0019; A61B 17/00491; A61L 26/0066; A61B 2017/00495; A61L 2300/202; A61L 2300/404

(57) ABSTRACT

The present invention is directed to systems for applying a silicone based topical skin adhesive, from a dual barrel syringe containing a two-part rapidly cross-linkable silicone composition for application onto tissue, the dual barrel syringe terminates in a static mixer configured for mixing and expressing the silicone composition, the static mixer having a medicant deposited on an inner surface thereof, the medicant configured for being released and picked up from the inner surface by the cross-linkable silicone composition during expression of the composition from the dual barrel syringe.

### FIELD OF THE INVENTION

The present patent application is generally related to medical devices and is more particularly related to dual barrel syringes that are used for dispensing rapidly reactive or polymerizable or cross-linkable flowable materials, particularly to mixing syringes suitable for expressing topical skin adhesives containing anti-microbial agents or other medicants that are incorporated into the mixture during mixing or immediately after mixing of the flowable materials.

### BACKGROUND OF THE INVENTION

In order to protect patients and enhance their post-procedure healing and recovery, it is important to prevent microbial colonization of the wound and surrounding tissue. Specifically in the field of topical Skin Adhesives and Wound Closure Systems, there is a need to provide an antimicrobial agent capable of diffusing onto a wound for prevention and mitigation of wound infections. There have been many efforts directed to providing antimicrobial agents for use with topical skin adhesives.

Further, there is a need to sterilize the contents of the medical delivery device, i.e. the medicant contained therein, with a number of sterilization techniques available, including thermal, gamma irradiation, e-beam, etc. A widely used sterilization technique entails exposure to Ethylene Oxide (EtO or EO) gas.

In one approach, a medicant, e.g. antimicrobial agent such as triclosan, can be added to the liquid skin adhesive (or one or both components of two-part liquid skin adhesive. However in some cases the medicant can react with the components of the adhesive (such as catalyst) during storage and or sterilization. Another potential disadvantage is that during the sterilization, the agent can pressurize the storage syringe, resulting in leaks.

In another approach, the medicant can be coated on a mesh that is positioned on the wound and then a liquid adhesive is applied over such mesh. This approach requires a special formulation of the mesh with incorporation of the medicant therein.

U.S. Publication No. 2021/0369258, entitled Systems, Devices And Methods For Dispensing And Curing Silicone Based Topical Skin Adhesives, discloses a system for dispensing a curable composition comprising: a delivery device; a static mixer having a proximal end that is connected with a distal end of said delivery device; a flexible spreader having a proximal end that is connected with a distal end of said static mixer, a distal end including a flat dispensing opening, and a plurality of channels extending through said flexible spreader to said flat dispensing opening.

U.S. Pat. No. 8,282,959, entitled Branched End Reactants And Polymeric Hydrogel Tissue Adhesives Therefrom, discloses a kit with a double barrel syringe that dispersion may optionally include at least one antimicrobial agent such as triclosan.

U.S. Pat. No. 10,531,949, entitled Silicone Foam Compositions Rapidly Cross-Linkable At Ambient Temperatures And Methods Of Making And Using Same, discloses a method of adjusting the volume of an implanted medical device in situ, comprising: providing a dual syringe hypodermic device comprising: a static mixer including a first syringe containing a catalyst fluid, a second syringe containing a crosslinker fluid, and an 18 to 21 gauge hypodermic needle, the static mixer configured to mix and inject the catalyst fluid and the cross-linker fluid substantially simultaneously at point of use; the catalyst fluid comprising a first low viscosity vinyl terminated polydimethylsiloxane present in an amount of from about 97 wt % to about 99.5 wt % based on the weight of the catalyst fluid and having a viscosity of from about 1 to about 150 cPs, and a platinum divinyl disiloxane complex comprising Pt [(CH₂═CH)(CH₃)₂SiOSi(CH₃)₂(CH═CH₂)]₃ having a platinum content of from about 2 ppm to about 32 ppm based on the catalyst fluid, wherein the catalyst fluid has a viscosity of from about 1 to about 150; and the cross-linker fluid comprising a second low viscosity vinyl terminated polydimethylsiloxane present in an amount of from about 1 wt % to about 40 wt % based on the weight of the cross-linker fluid and having a viscosity of from about 1 to about 150 cPs, a low viscosity hydride terminated polydimethylsiloxane present in an amount of from about 60 wt % to about 90 wt % based on the weight of the cross-linker fluid and having a viscosity of from about 1 to about 150 cPs, and a silicone cross-linker comprising polymethylhydrosiloxane polydimethylsiloxane copolymer present in an amount of from about 0.32 wt % to about 5.0 wt % based on the weight of the cross-linker fluid, and having a viscosity of from about 1 to about 150 cPs, wherein the cross-linker fluid has a viscosity of from about 1 to about 150 cPs, and wherein post-preparation or post-mixing of the catalyst fluid and the cross-linker fluid, a resultant rapidly cross-linkable silicone composition has a viscosity of ≤150 cPs for ≥1 min. post-preparation and ≤300 cPs≤5 min. post-preparation, at ambient temperature, and the resultant rapidly cross-linkable silicone composition has a platinum content of from about 1 ppm to about 16 ppm; mixing the catalyst fluid with the cross-linker fluid via the static mixer to produce an injectable composition having an initial viscosity of <150 cPs for at least about 1 min.; and within ≤5 min. of initiating mixing, and substantially simultaneous with mixing, injecting a predetermined volume of the injectable composition via the dual syringe hypodermic device, into the implanted medical device in situ, whereby the rapidly cross-linkable silicone composition substantially cross-links and gels in situ in an amount of time≥5 min. to produce a filled implant comprising a homogeneous silicone gel, wherein the rapidly cross-linkable silicone gel has a viscosity≥50,000 cPs≤24 hrs. post-injection at ambient temperature, and the volume of the implanted device has been adjusted.

U.S. Pat. No. 10,533,074, Silicone Compositions Rapidly Cross-Linkable At Ambient Temperatures And Methods Of Making And Using Same, discloses a method of filling an implanted medical device in situ, comprising: providing a two-part rapidly cross-linkable silicone composition, comprising: a catalyst fluid comprising a first low viscosity vinyl terminated polydimethylsiloxane present in an amount of from about 97 wt % to about 99.5 wt % based on the weight of the catalyst fluid and having a viscosity of from about 1 to about 150 cPs, and a platinum divinyl disiloxane complex comprising Pt [(CH₂═CH)(CH₃)₂SiOSi(CH₃)₂(CH═CH₂)]₃ having a platinum content of from about 2 ppm to about 32 ppm based on the catalyst fluid, wherein the catalyst fluid has a viscosity of from about 1 to about 150; and a cross-linker fluid comprising a second low viscosity vinyl terminated polydimethylsiloxane present in an amount of from about 1 wt % to about 40 wt % based on the weight of the cross-linker fluid and having a viscosity of from about 1 to about 150 cPs, a low viscosity hydride terminated polydimethylsiloxane present in an amount of from about 60 wt % to about 90 wt % based on the weight of the cross-linker fluid and having a viscosity of from about 1 to about 150 cPs, and a silicone cross-linker comprising polymethylhydrosiloxane polydimethylsiloxane copolymer present in an amount of from about 0.32 wt % to about 5.0 wt % based on the weight of the cross-linker fluid, and having a viscosity of from about 1 to about 150 cPs, wherein the cross-linker fluid has a viscosity of from about 1 to about 150 cPs, and wherein post-preparation or post-mixing of the catalyst fluid and the cross-linker fluid, the resultant rapidly cross-linkable silicone composition has a viscosity of ≤150 cPs for ≥1 min. post-preparation and ≤300 cPs≤5 min. post-preparation, at ambient temperature, and the resultant rapidly cross-linkable silicone composition has a platinum content of from about 1 ppm to about 16 ppm; mixing the catalyst fluid with the cross-linker fluid to produce an injectable composition having an initial viscosity of <150 cPs for at least about 1 min.; and within ≤5 min. of initiating mixing, injecting a predetermined volume of the injectable composition into the implanted medical device in situ, whereby the rapidly cross-linkable silicone composition substantially cross-links and gels in situ in an amount of time≥5 min. to produce a filled implant comprising a homogeneous rapidly cross-linkable silicone gel, wherein the rapidly cross-linkable silicone gel has a viscosity≥50,000 cPs≤24 hrs. post-injection at ambient temperature.

U.S. Pat. No. 6,698,622, Double-Barreled Syringe With Detachable Locking Mixing Tip, discloses a syringe for dispensing two viscous materials as an admixture, comprising: an integrally molded housing having a first and a second barrel symmetrically opposed along a center line, the barrels are enclosed at a discharge end and at an inlet end, have a generally cylindrical neck extending from and symmetrically disposed at the discharge end along the center line, the neck including first and second outlet passages in fluid communication, respectively, with the first and second barrels, an exterior circumferential surface defining the shape of the barrels, and a gripping member disposed at the inlet end of the housing; a first mating assembly having opposed first and second locking ribs symmetrically disposed with respect to said neck and rigidly attached to the enclosed discharge end, the first and second locking ribs having at least a portion aligning with the exterior circumferential surface of the two barrels, and wherein the first mating assembly is configured to receive a mixing tip or a locking closure cap; wherein the mixing tip has an inlet, an outlet, a bore comprising a neck receiving section defined by a circumferential surface adapted to closely receive the neck, and a second mating assembly having opposed generally planar first and second locking tabs symmetrically disposed with respect to the bore, the two mating assemblies conjoining when the neck is inserted into the mixing tip bore in a relative orientation, and wherein the two mating assemblies interlock when the mixing tip rotates in a first direction and the locking tabs slide under the first and the second locking ribs until a stop is achieved, the two mating assemblies are detachable when the mixing tip is rotated in an opposite direction; and wherein the mixing tip further including a static mixing element having a plurality of intertwined sections disposed within the bore of the mixing tip for mixing the two viscous materials as the two viscous materials are dispensed from the first and the second barrels.

U.S. Publication No. 2012/0295214, entitled Dental Auto-Mixing Methods, Devices, And Compositions, discloses a method of dispensing a hardenable dental composition comprising: providing a multi-part hardenable dental composition comprising: a part (A) in the form of a paste, comprising: acid-reactive glass particles and a liquid selected from the group consisting of water, a monomer having at least one ethylenically unsaturated group per monomer molecule, and a combination thereof; and a part (B) comprising: a water miscible polyacid and a liquid selected from the group consisting of water, a monomer having at least one ethylenically unsaturated group per monomer molecule, and a combination thereof; wherein: water is included in part (A); part (B); or parts (A) and (B); the monomer having at least one ethylenically unsaturated group per monomer molecule is included in part (A); part (B); or parts (A) and (B); and at least one component for initiating polymerization of the monomer is included in part (A); part (B); or parts (A) and (B); and extruding the composition through a static mixer in fluid communication with a first reservoir containing the part (A) and a second reservoir containing the part (B); wherein a plunger is positioned in each reservoir for simultaneously forcing part (A) and part (B) into the static mixer, extruding the composition through the static mixer, and dispensing the composition; and wherein an extrusion force of less than 40 pound-force (178 newtons) according to Test Method I is applied to the plunger for extruding the composition through the static mixer without the aid of a mechanical advantage provided by an attached or external device.

In spite of the above advances, there is a continuing need for systems, devices, and methods for easily and efficaciously adding a medicant to a rapidly polymerizable topical skin adhesives, in particular systems that allow for sterilizability and storage of the medical devices and materials.

### SUMMARY OF THE INVENTION

In one aspect, this invention relates to a system for applying a silicone based topical skin adhesive, comprising: a dual barrel syringe containing a two-part rapidly cross-linkable silicone composition for application onto tissue, the dual barrel syringe terminating in a static mixer configured for mixing and expressing the silicone composition, the static mixer having a medicant deposited on an inner surface thereof, the medicant configured for being released and picked up from the inner surface by the cross-linkable silicone composition during expression of said composition from said dual barrel syringe. In certain embodiments the medicant comprises triclosan or chlorhexidine gluconate or silver compound and is deposited with or onto a binder.

The binder is soluble in the silicone composition and can comprise high molecular weight non-crosslinked methyl terminated polydimethylsiloxane (PDMS), or partially cross-linked PDMS.

In one aspect, the medicant is configured for being released during expression of the composition predominantly at the beginning of expression or alternatively predominantly uniformly from the beginning to the end of expression.

In another aspect, this invention relates to a kit comprising the system for applying a silicone based topical skin adhesive and a flexible porous mesh configured for application onto the tissue and for approximating edges of a wound.

In yet another aspect, this invention relates to a method of treating a wound comprising the steps: Directing towards the tissue the dual barrel syringe; Expressing from the dual barrel syringe the silicone composition through the static mixer resulting in the medicant being incorporated into the composition, Coating the tissue or a mesh positioned on the tissue with the silicone composition containing the medicant; Allowing the silicone composition to cure.

### BRIEF DESCRIPTION OF THE DRAWINGS

FIG. 1 shows a schematic cross-sectional view of a dual barrel syringe.

FIGS. 2A, 2B show images of agar plates with mesh for ZOI measurements of Example 1.

FIGS. 3A, 3B show images of agar plates with mesh for ZOI measurements of Example 1.

FIGS. 4A, 4B show images of agar plates with mesh for ZOI measurements of Example 1.

FIGS. 5A, 5B show images of agar plates with mesh for ZOI measurements of Example 1.

FIGS. 6A, 6B show images of agar plates with mesh for ZOI measurements of Example 1.

FIGS. 7, 8 show images of control agar plates of Example 1.

FIG. 9 shows results corresponding to Example 3.

### DETAILED DESCRIPTION OF THE PREFERRED EMBODIMENTS

Briefly, according to the present invention, in some embodiments, a medicant that is beneficial for wound healing, such as an antimicrobial agent (such as triclosan, chlorhexidine gluconate, silver salts, polyhexamethylene biguanide (PHMB), and combinations thereof) is deposited inside the expression tip or the static mixer of a two-part liquid adhesive syringe. Preferably, the medicant or agent is incorporated into a binder, such as silicone based binder, deposited inside the mixer. As the liquids are expressed and flow through the static mixer and or the expression tip, the liquids interact with the medicant and picks up the medicant and carries it onto the wound. This system is different to the medicant being deposited onto the dressing and/or mesh and/or directly added to the liquid composition inside the syringe.

Advantageously, having the medicant in a dried coating form simplifies the assembly, storage, and sterilization of the devices, and prevents any reactivity between the medicant and the components of the liquid adhesive during long term storage, and during sterilization. Advantageously, reactivity with the topical skin adhesive components, such as cross-linker, catalyst, etc., is minimized as there is only a short term contact between the medicant and the liquid composition prior to the curing and solidifying of the composition into a on-wound dressing, cover, or patch.

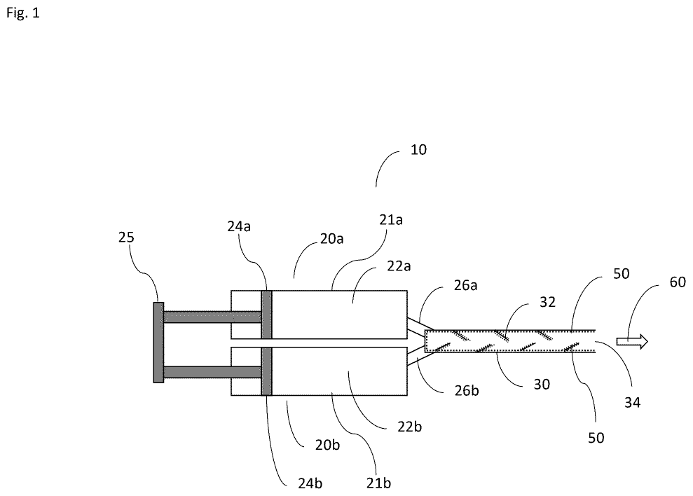

Referring to FIG. 1, an embodiment of the present invention is shown. Dual barrel syringe 10 comprises a first syringe 20a and a second syringe 20b, joined together in a unified construct forming dual barrel syringe 10. Dual barrel syringe 10 is adapted for handling and simultaneous expression of first liquid component 22a and second liquid component 22b that are disposed in correspondingly first barrel 21a and second barrel 21b. First liquid component 22a and second liquid component 22b are reactive upon mixing and can form rapidly highly adhesive or bondable curable composition or topical skin adhesive that is curable in under 5 minutes, more preferably under 3 minutes on animal tissue, such as on skin.

First piston 24a and second piston 24b are slidably moveable inside correspondingly first barrel 21a and second barrel 21b upon action of unified handle 25. First barrel 21a and second barrel 21b are in fluid communication with hollow mixing and expression tip 30, though connectors 26a and 26b. Mixing and expression tip 30 has optional baffles 32 protruding into the interior of tip 30. Baffles 32 enhance mixing of liquids inside tip 30 due to turbulence of flow and tortuous path inside tip 30 thus improving static mixing inside tip 30 of first liquid component 22a and second liquid component 22b as these components advance from syringe barrels to be expressed from tip 30 as a mixture, schematically shown by arrow 60.

At least a portion of inner surface of tip 30 is coated by coating 50, the coating comprising a beneficial medicant or agent. Coating 50 is schematically shown by a dotted line inside tip 30. Coating 50 is soluble or abradable when in contact with first liquid component 22a and second liquid component 22b.

In use, expression of first liquid component 22a and second liquid component 22b is effected by first piston 24a and second piston 24b, the pistons advancing upon action of handle 25. Upon advancing pistons 24a and 24b in barrels 21a and 21b, first liquid component 22a and second liquid component 22b are moving through first connector 26a and second connector 26b and into mixing and expression tip 30. Inside tip 30 liquid components 22a and 22b mix and start reacting, but do not fully cure due to rapid movement of liquid components 22a and 22b through tip 30 to avoid clogging of tip 30 by fully cured topical skin adhesive forming upon mixing of liquid components 22a and 22b. Expression of fully mixed but not fully cured topical skin adhesive from open distal end 34 of tip 30 is shown by arrow 60.

Advantageously, as liquid components 22a and 22b advance through tip 30, the liquid is interacting with and is dissolving in itself medicant 50 that is coated on inner surface of tip 30. Presence of optional baffles 30 helps to increase surface are and quantity of medicant contained inside tip 30. Baffles 30 help to increase uniform mixing and homogenization of liquid components 22a and 22b as well as uniform mixing and homogenization of medicant from coating 50 within liquid components 22a and 22b mix.

Upon exiting open distal end 34 of tip 30 liquid components 22a and 22b have mixed and formed topical skin adhesive that also contains medicant dissolved therein, the flow of the topical skin adhesive indicated by arrow 60.

A number of methods can be utilized to deposit medicant or agent inside tip 30 to form coating 50. In one embodiment, a solution of medicant in a liquid solvent is applied by spray or dip coating onto inside surface of tip 30, or is pumped through tip 30. The solution is then allowed to dry, leaving on the inside surface of tip 30 coating 50 containing medicant. In a preferred embodiment, a solution of medicant and non-volatile excipient in a volatile solvent is utilized. The solvent is allowed to evaporate leaving on the surface of tip 30 coating 50 containing medicant and excipient.

In preferred embodiments, the agent is deposited as a liquid, comprising a volatile solvent, the medicant, and an optional excipient or medicant binder, whereby after coating of inside surface of tip 30, evaporation of the solvent is executed, by drying, performed at ambient conditions, or optionally under vacuum and/or elevated temperature, leaving behind the medicant/agent and the optional excipient. For example, a solution of Triclosan in ethyl acetate or ethanol, with optional excipients, can be used. Such solution can have some of non-volatile excipient or binder in it, to absorb more triclosan and keep it on the walls of the static mixer and/or expression tip 30 after the solvent evaporates.

In one embodiment, the medicant is triclosan and the binder facilitating attachment of the medicant onto the inner surface of the static mixer in tip 30 comprises high molecular weight non-cross-linkable polydimethylsiloxane. The binder is preferably trimethylsilyl-terminated polydimethylsiloxane, which is a linear high molecular weight polydimethylsiloxane polymer, and which contains no reactive functions. This polymer provides a non-cross-linked phase in the resulting silicone coating inside tip 30, and is believed to form a matrix phase to incorporate and hold the medicant/active agent within it. In some embodiments, the weight average molecular weight of high molecular weight non-cross-linkable polydimethylsiloxane polymer used as a binder is from about 300,000 to about 15,000,000, preferably between about 400,000 to about 700,000. Examples of the polymer used as a binder include, but are not limited to: Gelest Product Code No. DMS-D-56, DMS-T62, DMS-T61, DMS-D72, and similar.

The binder material is preferably in a form of solid which is easily releasable and/or dissolvable and/or abradable from the surface of static mixer by passing liquids during mixing action.

In certain embodiments, the medicant, such as triclosan, is deposited as a vapor by exposing the static mixer inside tip 30 to evaporating or sublimating triclosan. Optionally, the surface of the static mixer can be pre-coated with the binder to facilitate the deposition of triclosan from the vapor phase.

### Example 1

Triclosan as a medicant was dissolved in liquid Ethyl Acetate (EtAc) together with polydimethylsiloxane (PDMS) as an binder or excipient and then coated inside a static mixer, forming a labile coating inside the static mixer. Then a cross-linkable silicone formulation was expressed through the static mixer whereby triclosan with PDMS was picked up by the cross-linkable silicone formulation upon expression through the static mixer. The expressed and rapidly cross-linking formulation was expressed onto a polymeric mesh, coating the mesh and solidifying on the mesh. The mesh coated with the silicone coating was then analyzed microbiologically for presence of antimicrobial medicant triclosan. The analysis was performed by visualizing the Zone of Inhibition (ZOI) around the silicone coated mesh. The mesh was positioned onto an agar filled Petri dish, in a center thereof, whereby agar was inoculated with a model bacterial strain. The presence of triclosan is indicated by ZOI which is a zone with no bacterial growth around the mesh.

Rapidly cross-linkable two-part silicone formulation or TSA (topical skin adhesive) was prepared using formulation as shown below:

### Part A:

90 g of Elkem 55 experimental base (also known as Elkem Silbione 4020-55, containing vinyl terminated polydimethyl silicone base polymer and fumed silica particles) was mixed with 10 g of Example 1-SF (Outlined in U.S. patent application Ser. No. 18/347,211, [55]), using a high-speed centrifugal mixer (FlackTek DAC150 FV-K) at 3470 rpm for 3 minutes. This composition had a viscosity of 32,090 cPs.

### Part B

90 g of Elkem 55 experimental base was mixed with 9.0 g of polymethylhydro-co-polydimethyl siloxane cross linker (Gelest HMS H301), 3.0 g of SiH terminated polydimethylsiloxane chain extender (Gelest DMS H21) using a high-speed centrifugal mixer (FlackTek DAC150 FV-K) at 3470 rpm for 3 minutes. This composition had a viscosity of 31,160 cPs

10 g of the rapidly cross-linkable two-part silicone formulation shown above was mixed using a 2.25 cm static mixer (Sulzer MBH07-11D, the mixer portion of cut to stated length) and coated onto 10 cm PET porous mesh (PET mesh also had PSA (pressure sensitive adhesive) coated on polyester mesh (Lot #16204, Innovize, St Paul, MN). The mesh was pre-coated with pressure sensitive adhesive (PSA) on one side and adhered to a polyethene release liner prior to the silicone TSA application.

Five (5) types of triclosan coated static mixers were tested using this procedure. Small samples or coupons of the silicone TSA coated PET mesh were cut out and tested for ZOI. Two mesh coupons were cut for each type of static mixer, the first 1 cm (sample B or beginning) and the last 1 cm of the 10 cm PET mesh (sample A or end). The silicone TSA was applied onto the mesh from B (begin) to A (end) whereby all TSA was expressed onto the mesh.

The bacteria used on the Petri dishes was Bacteria: S. aureus ATCC 6538 Inoculum Count: 1.3×10\hat{ }5 CFU/plate.

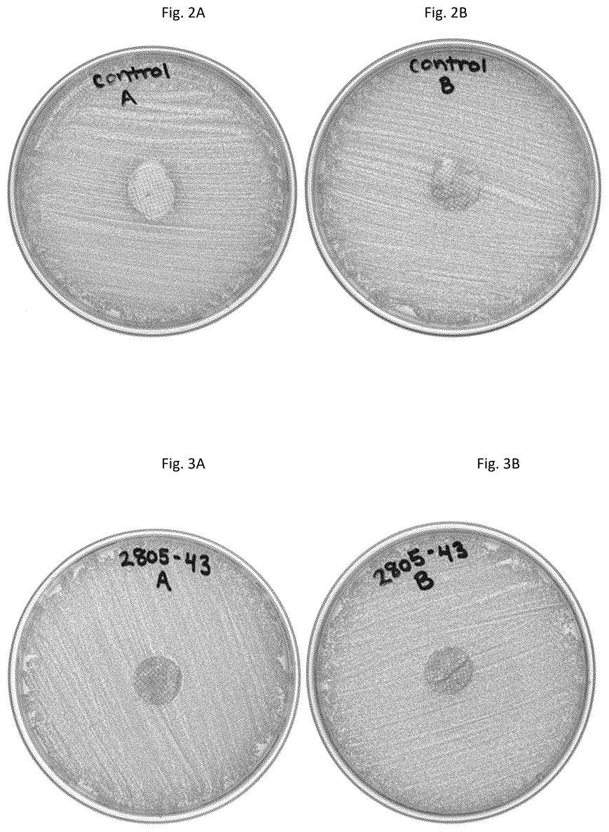

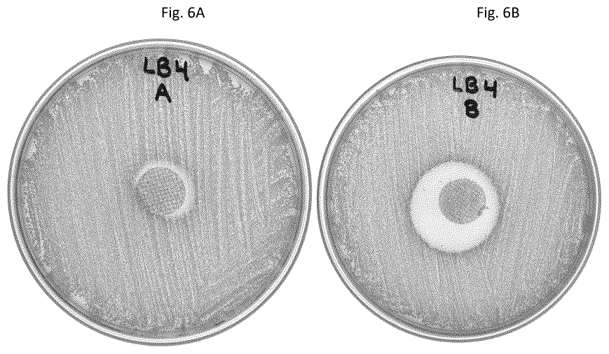

The descriptions of the tested systems and the results are presented in Table 1 and thereafter, as well as in referenced FIGS. 2-6 showing agar plates with mesh and ZOI.

TABLE 1
Tested samples and ZOI measurements

| | Sample and image | | | | | | | | | |
|---|---|---|---|---|---|---|---|---|---|---|
| | | | | | LB2: static | | LB3: static | | LB4: static | |
| | | | | | mixers coated | | mixers coated | | mixers coated | |
| | | | | | with 33.3% | | with 33.3% | | with 33.3% | |
| | Control: static | | 2805-43: | | triclosan 66.7% | | triclosan 66.7% | | triclosan 66.7% | |
| | mixer coated | | evaporative | | high Mw non- | | high Mw non- | | high Mw non- | |
| | with 50% | | deposition of | | cross linked | | cross linked | | cross linked | |
| | triclosan ethyl | | triclosan onto | | PDMS in EtAc, | | PDMS in EtAc, | | PDMS in EtAc, | |
| | acetate solution, | | static mixer no | | 14.3% solid | | 10.8% solid | | 8.7% solid | |
| | no binder used | | binder used | | content | | content | | content | |
| FIGS. | 2A | 2B | 3A | 3B | 4A | 4B | 5A | 5B | 6A | 6B |
| Tested area | A | B | A | B | A | B | A | B | A | B |
| of coated | | | | | | | | | | |
| mesh A = end; | | | | | | | | | | |
| B = beginning | | | | | | | | | | |
| ZOI around | 0.0 | 0.0 | 0.0 | 0.0 | 0.5 | 4.1 | 0.0 | 6.2 | 1 | 6.1 |
| the mesh | | | | | | | | | | |
| coupon (mm) | | | | | | | | | | |

The control sample was the static mixer coated with 50% triclosan ethyl acetate solution, with ZOI results shown in FIG. 2, indicating very limited or no ZOI.

2805-43 sample was prepared using the evaporative deposition of triclosan. The static mixer was placed inside a paper folder together with two Tyvex patches impregnated with the triclosan (double the amount of triclosan dose typically used in sutures coating). The entire package was Ethylene Oxide (EO) sterilized using cycle X. ZOI results are shown in FIG. 3, indicating very limited or no ZOI.

Inventive samples LB2, LB3, LB4 are static mixers coated with 33.3% triclosan and the binder which was 66.7% high molecular weight non cross linked polydimethylsiloxane (Gelest DMS T72, Molecular weight>500,000). Both solids were dissolved in ethyl acetate together, the concentrations of triclosan/DMS T72 mixture in ethyl acetate solutions were varied from 8.7 to 14.3 percent by weight.

In some embodiments, medicant to binder ratio by weight is between 1-1.5 to 1-20, preferably between 1 to 4.

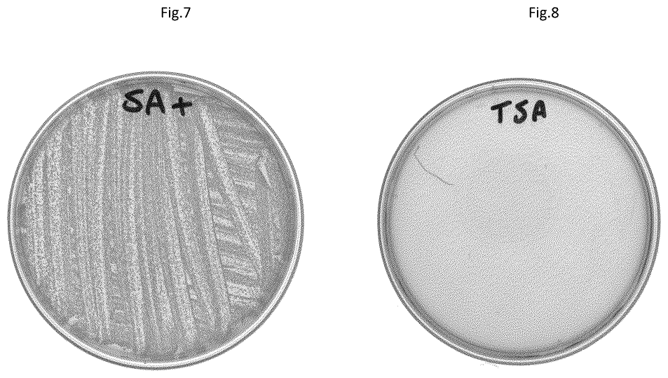

Additional control testing results are shown in FIGS. 7 and 8. FIG. 7 shows further negative control image of agar plate-Petri dish with no mesh applied and showing no ZOI whatsoever. FIG. 8 shows further positive control image of agar plate-Petri dish with TSA containing excess amount of triclosan and showing complete microbial elimination (i.e. ZOI which is larger than the Petri dish).

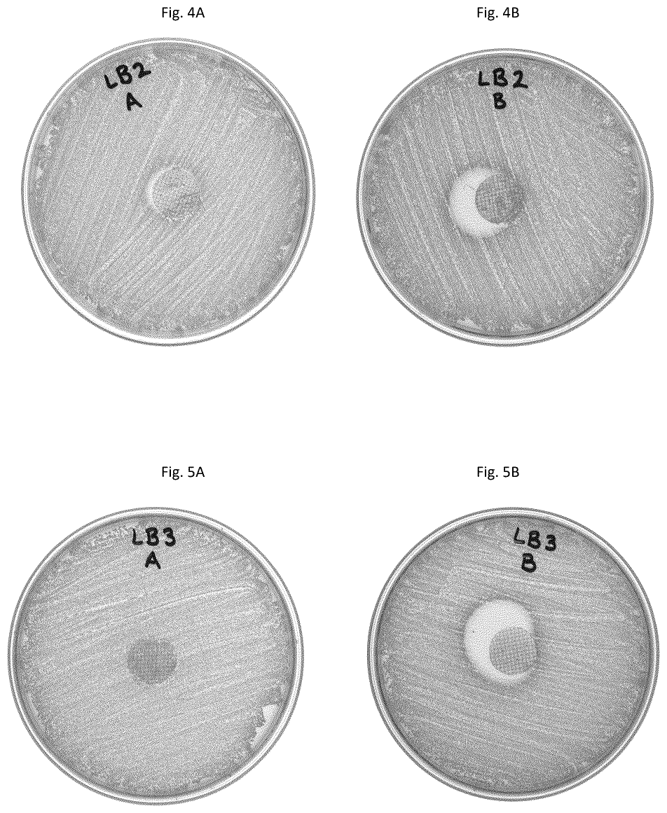

The inventive embodiments are corresponding to ZOI results on Agar plates with meshes coated after TSA expressed from the static mixers (coated with triclosan) are shown in referenced FIGS. 4, 5, 6, indicating substantial ZOI especially at the beginning of expression, with some ZOI also at the end of the expression.

Clearly using non-crosslinked PDMS silicone binder to load triclosan shows positive results, with significant triclosan loading and excellent pickup of the deposited medicant. There is a variability between early expressed material and later expressed material, but a significant ZOI is observed. The reason is the different rate of pickup of the excipient/binder containing triclosan. By varying the binder viscosity and solubility as well as quantity, the triclosan pickup rate can be made more uniform. In some embodiments, two types of non-cross linked PDMS, one with high molecular weight (>500,000, Gelest DMS T72), one with relatively lower Mw (300,000, Gelest DMS T53) which will make the release more uniform. In some embodiments, PDMS is partially cross-linked to make the release more uniform. In some embodiments, the binder contains other agents (chlorhexidine gluconate CHG, etc.).

By performing further design optimization, amount of triclosan on the static mixer, length of the mixer, viscosity of the liquids expressed, a range of medicant expression quantity, uniformity, etc. can be achieved. In come embodiments, it could be beneficial to express most of medicant at the beginning of expression, with the early expressed TSA (containing most medicant) applied onto the wound area, and later expressed TSA (containing none or smaller loadings of medicant) applied on a periphery of the wound, where the medicant is less beneficial or not needed. In alternative embodiments, a more uniform expression of medicant can be desirable and achievable.

### Example 2

Further testing and optimization was performed with different static mixers and length of medicant coating. In the Table 2 presented below ZOI results are presented for various T72 and T53 static mixers that were coated using 2-5 cm mixing tip length with medicant triclosan coating. TSA with variable viscosity of 20k-60k cPs was used in the testing. Meshes were coated similarly to Example 1 and ZOI evaluated on agar plates.

TABLE 2
Tested samples and ZOI measurements

| | Static mixer | | | |
|---|---|---|---|---|
| | type, static mixer | | | |
| | length (the | ZOI (mm) S. aureus | ZOI (mm) S. aureus | |
| | whole length | Mesh coated at the | Mesh coated in the | ZOI (mm) S. aureus |
| TSA | was coated with | beginning of | middle of | Mesh coated at the |
| viscosity | medicant | expression | expression | end of expression |
| 30k cPs | T72, 3 cm | 6.2 | 0.0 | 0.0 |
| 20k cPs | T72, 2 cm | 0.0 | 0.0 | 0.0 |
| 20k cPs | T72, 3 cm | 0.0 | 0.0 | 0.0 |
| 20k cPs | T72, 4 cm | 0.0 | 0.0 | 0.0 |
| 20k cPs | T72, 5 cm | 23 | 19.7 | 0.0 |
| 60k cPs | T53, 2 cm | 0.0 | 0.0 | 0.0 |
| 60k cPs | T53, 3 cm | 21.2 | 21.4 | 21.3 |
| 60k cPs | T53, 4 cm | 22.5 | 24.4 | 21.7 |
| 60k cPs | T53, 5 cm | 25.5 | 24.8 | 25.1 |

As seen in Table 2, a uniform expression of TSA form beginning to end of the expression, can be achieved at specific optimized parameters of viscosity of TSA and length. All 60k cPs at 3, 4, 5 cm were uniform expression of medicant from beginning to end. 2 cm length was not showing any ZOI.

### Example 3

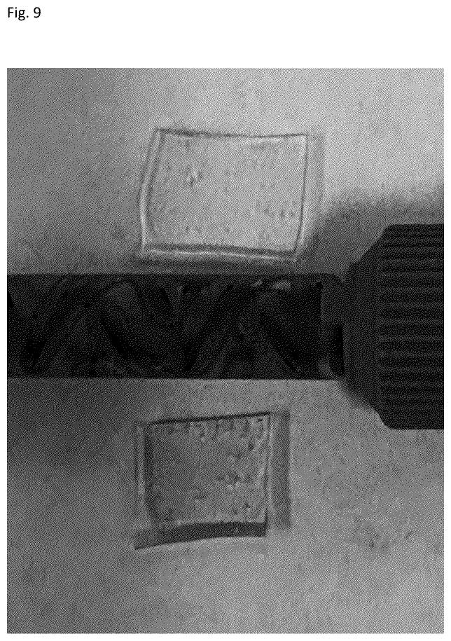

Referring to FIG. 9, the silicone based TSA as described above was expressed through a dye-containing static mixer for visualization. The dye was deposited on the inside walls of the static mixer to show the mechanism of the transfer of materials from the mixer and to TSA. FIG. 9 shows the dye-containing static mixer (middle) and a cured sample of TSA expressed through such static mixer (bottom), showing coloration due to the dye which was picked up by TSA from the mixer. On top of FIG. 9 is shown the cured silicone TSA which was expressed through a regular, non-dye containing static mixer (not shown), as a control. No coloration is observed. The example visually indicates how additional components or ingredients, such as beneficial medicants, can be added through deposition on the inside wall of the expression tip and or static mixer.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

I/We claim:

1. A system for applying a silicone based topical skin adhesive, comprising:
a) A dual barrel syringe having at least two barrels, each barrel having piston rods with plungers that are responsive to an expression force, each separately containing different reactive components that form a rapidly cross-linkable silicone composition for application onto tissue,
b) each barrel of the dual barrel syringe having at a terminus end a fluid outlet and conduit to a static mixer configured to receive the reactive components from both barrels and for conveying and mixing the reactive components of the silicon composition from both barrels,
wherein
(i) the static mixer has a releasable medicant coating on an inner surface thereof, and
(ii) the releasable medicant releases from the inner surface as the reactive components for the silicon composition are conveyed due to the expression force through the static mixer to the mixer outlet.

2. The system of claim 1, wherein the medicant comprises triclosan or chlorhexidine gluconate or silver compound and is deposited with or onto a binder.

3. The system of claim 2, wherein said binder is soluble in the silicone composition.

4. The system of claim 3, wherein said binder comprises high molecular weight non-crosslinked methyl terminated polydimethylsiloxane (PDMS), or partially cross-linked PDMS.

5. The system of claim 4, wherein said binder preferably comprises a non-cross-linked high molecular weight PDMS with molecular weight over 200,000.

6. The system of claim 4, wherein the medicant to binder ratio by weight is between 1-1.5 to 1-20, preferably between 1 to 4.

7. The system of claim 4, wherein said medicant is deposited onto the inner surface by coating the inner surface with a solution of PDMS and triclosan in volatile solvent.

8. The system of claim 7, wherein said volatile solvent is ethyl acetate.

9. The system of claim 1, wherein the length of the static mixer is greater than 2 cm.

10. The system of claim 1, wherein greater amounts of medicant are released with an initial portion of blended reactive components for the silicone composition that is conveyed through the static mixer.

11. The system of claim 1, wherein predominantly uniform amounts of medicant are released with an entire portion of the blended reactive components for the silicone composition is conveyed through the static mixer.

12. A kit comprising the system of claim 1 and a flexible porous mesh configured for application onto the tissue and for approximating edges of a wound.

13. The kit of claim 12, wherein said mesh has a pressure sensitive adhesive disposed on one side thereof.

14. A method of making the system of claim 1, comprising the steps of:
(i) Filling the barrels of said dual barrel syringe with the two-part rapidly cross-linkable silicone composition;
(ii) Depositing the medicant onto the inner surface of the static mixer.

15. A method of treating a wound using the system of claim 1, comprising the steps:
(i) Directing towards the tissue the dual barrel syringe,
(ii) Expressing from the dual barrel syringe the silicone composition through the static mixer resulting in the medicant being incorporated into the composition,
(iii) Coating the tissue or a mesh positioned on the tissue with the silicone composition containing the medicant;
(iv) Allowing the silicone composition to cure.
